(19) United States
(12) Patent Application Publication
(10) Pub. No.: US 2004/0141916 A1
(43) Pub. Date: Jul. 22, 2004

(54) Prevention of systemic toxicity during radioimmunotherapy for intravascularly disseminated cancers

(76) Inventor: George Sgouros, Ellicott City, MD (US)

Correspondence Address: Benjamin Aaron Adler, ADLER & ASSOCIATES, 8011Candle Lane, Houston, TX, 77071, US

(21) Appl. No.: 10/753,141

(22) Filed: Jan. 7, 2004

Related U.S. Application Data
(60) Provisional application No. 60/438,747, filed on Jan. 8, 2003.

(51) Int. Cl. A61K 51/00

(52) U.S. Cl. 424/1.49

(57) ABSTRACT

The present invention provides a method to target delivery of antibody constructs radiolabeled with an alpha-particle emitting radionuclide to intravascularly disseminated tumor cells while minimizing systemic toxicity. Targeted delivery of a construct specific for a protein expressed on the tumor cells in combination with rapid clearance of intravascular non-targeted radiolabeled antibody construct minimizes alpha particle emissions from the radionuclide and its alpha-particle emitting decay intermediates to non-targeted cells.

### CROSS-REFERENCE TO RELATED APPLICATION This non-provisional application claims benefit of provisional U.S. Serial No. 60/438.747, filed Jan. 8, 2003, now abandoned.

### BACKGROUND OF THE INVENTION

1. Field of the Invention

This invention relates generally to the fields of radioimmunotherapy and cancer treatment. More specifically, this invention relates to targeted delivery of actinium-225-labeled antibodies and the removal of non-targeted actinium-225-labeled antibodies from the blood.

2. Description of the Related Art

The humanized monoclonal antibody, Herceptin, directed against HER-2/neu has, in combination with chemotherapy, been effective in the treatment of breast cancer malignancies overexpressing HER-2/neu. The HER-2/neu oncogene encodes a transmembrane protein (p185^{HER2}) with extensive homology to the epidermal growth factor (EGF) receptor. Amplification and overexpression of HER-2/neu have been documented in many human tumors, most notably in breast cancer. The expression of HER-2/neu is relatively stable over time and is generally congruent at different metastatic sites.

However, HER-2/neu protein has also been identified on cell membranes of epithelial cells in the gastro-intestinal, respiratory, reproductive, and urinary tract as well as in the skin, breast and placenta. HER-2/neu expression levels in these normal tissues are similar to the levels found in non-amplified, non-overexpressing breast cancer cells. Approximately 30% of breast cancer patients have tumors overexpressing the HER-2/neu receptor. Herceptin treatment has been limited to these patients because of the cross-reactivity with normal tissues noted above. HER-2/neu has been previously considered for radioimmunotherapy against breast cancer. The beta emitter, I-131, and also Pb-212, whose daughter, Bi-212, decays by alpha particle emission, have been labeled to antibodies targeting HER-2/neu and investigated in animal models.

The alpha-particle emitting atomic generator, ²²⁵Ac has a 10-day half-life. Each decay of ²²⁵Ac leads to the emission of four alpha particles with initial energies in the range of 5-8 MeV, as depicted in FIG. 1, thereby greatly increasing its efficacy over previously considered alpha-particle emitters. Studies in vitro and in animal models have shown that this radionuclide is approximately 1000-fold more effective per unit radioactivity than ²¹³Bi, a first generation alpha-emitter that is currently under clinical investigation. Studies in animals, however, have also shown that, depending upon the administration route, target and chelation chemistry, ²²⁵Ac can be substantially more toxic.

The increased efficacy arises because ²²⁵Ac, with a longer half-life of 10 days vs. 45.6 min for ²¹³Bi, increases the total number of decays per unit radioactivity and allows prolonged irradiation of targeted cells. Furthermore, ²²⁵Ac decay leads to the release of three alpha-particle-emitting daughters. Including the parent ²²⁵Ac, a total of 4 alpha particles are emitted per ²²⁵Ac decay to a stable nuclide.

The toxicity arises because antibody delivery of this radionuclide can only deliver the first of the four alphas. The bond between the targeting vehicle and the radionuclide is broken upon transformation of the parent and emission of the first alpha; subsequent alpha-emitting daughters are, therefore, free to distribute throughout the body and potentially irradiate normal organs. This can be mitigated if the radiolabeled antibody is internalized since charged daughter atoms produced intracellularly are retained within the cell (McDevitt, et al. Science (2002)).

However, Herceptin-mediated targeting of ²²⁵Ac to disseminated breast cancer, or to any alpha particle emitting radiolabeled antibody targeted to a disseminated cancer, would present a problematical therapeutic approach in humans due to the high background expression of HER-2/neu or of other expressed genes in normal tissues. Such targeting is likely to lead to alpha-particle irradiation of normal crossreactive tissues and to the potential toxicity associated with the distribution of free, alpha-particle emitting daughters that result from the decay of ²²⁵Ac or of ²²³Ra. These drawbacks may be obviated by targeting rapidly accessible micrometastatic disease in a treatment schedule in which intravenously administered ²²⁵Ac-Herceptin or other alpha particle-emitting radionuclide is allowed to distribute for several hours and is then cleared from the circulation, either by direct physical means, such as extracorporeal immunoadsorption (Wang et al. Cancer. 94 (4 Suppl), pp. 1287-92 (2002)), or by administration of secondary clearing agents (Paganelli G, Chinol M, Maggiolo M, et al. Eur J Nucl Med. 24(3), pp. 350-1 (1997;)). Extravasation of intact antibody into normal tissue parenchyma generally requires 24 to 48 hours (Pimm et al. Nucl Med Commun. 10(8), pp. 585-93 (1989)).

Extracorporeal immunoadsorption involves the use of an affinity column that binds to and retains radiolabeled antibody. The patient's plasma volume is typically passed through this column to reduce the amount of radiolabeled antibody in the circulation. Similarly, the clearing agent approach reduces the concentration of radiolabeled antibody in the circulation. This is accomplished by intravenous administration of an agent that complexes with the antibody leading to catabolism and excretion of the radiolabel. Since excretion generally occurs via the kidneys, the radiation dose to the kidneys is increased and can potentially become dose-limiting.

These approaches have been used to target disease that is extravascular, requiring prolonged radiolabeled antibody circulation in order to target the disease. By rapidly decreasing the concentration of circulating antibody, the amount of antibody binding to normal cross-reactive tissues would be reduced substantially. Concomitantly the 225Ac concentration in the circulation is reduced and, therefore, the subsequent concentrations of alpha particle-emitting free daughters.

The inventors have recognized a need for an effective means of targeting intravascularly disseminated tumor cell clusters for radioimmunotherapy while minimizing toxicity resulting from the therapy. Specifically, the prior art is deficient in the lack of an effective means of targeting intravascularly disseminated breast cancer metastases with 225Ac-labeled Herceptin with a concomitant need for rapidly clearing non-targeted intravascular 225Ac-labeled Herceptin. The present invention fulfills this long-standing need and desire in the art.

### SUMMARY OF THE INVENTION

In one embodiment of the present invention, there is provided a method of preventing toxicity during radioimmunotherapy for intravascularly disseminated tumor cells in an individual. This method comprises the steps of administering an antibody construct labeled with an alpha-particle emitting radionuclide where the antibody is specific for a protein expressed in said intravascular tumor cells; rapidly targeting the radiolabeled antibody construct to the intravascular tumor cells; internalizing the radiolabeled antibody conjugate into the intravascular tumor cells; and rapidly clearing the non-targeted intravascular radiolabeled antibody construct. A combination of internalizing the radionuclide into the intravascular tumor cells and rapid clearance of the non- targeted intravascular radiolabeled antibody construct decreases alpha particle emission from the radionuclide and decay intermediates thereof to non-targeted cells thereby preventing systemic toxicity.

In another embodiment of this invention there is provided a method of targeting intravascularly disseminated breast cancer metastases for delivery of an alpha-particle emitting radionuclide thereto with minimal targeting of normal cells with the alpha-particle emitting radionuclide or alpha-particle emitting decay intermediates thereof in an individual, comprising the steps of conjugating the radionuclide with Herceptin antibody to form a radiolabeled Herceptin construct; administering the radiolabeled Herceptin construct intravenously to the individual; targeting the radiolabeled Herceptin construct to HER-2/neu protein expressed on the breast cancer metastases; internalizing the radiolabeled Herceptin construct into the breast cancer metastases; and rapidly clearing non-targeted intravascular radiolabeled Herceptin construct such that a combination of internalizing the radionuclide into the intravascular breast cancer metastases and rapid clearance of the non-targeted intravascular radiolabeled Herceptin construct minimizes targeting the alpha particle-emitting radionuclide and alpha-particle emitting decay intermediates thereof to normal cells.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts a simplified decay scheme for ²²⁵Ac. The arrows designate decay by alpha-particle emission with the average energy of emitted alpha-particles shown next to each arrow. The decay of ²¹³Bi Beta decays are omitted from the Figure, as is the short-lived daughter of Bi-213.

FIG. 2 depicts confocal microscopy images of spheroids (Φ˜200 mm) following 1, 3 and 5 hr incubation with 10 μg/ml Herceptin-FITC. The black or gray regions reflect presence of Herceptin. Individual cells are clearly outlined in the surface layer of MDA and BT spheroids, consistent with cell-surface localization of HER-2/nue. At 10 μg/ml Herceptin-FITC, no uptake of Herceptin was observed in MCF7 spheroids.

FIG. 3 depicts Herceptin concentration profiles across the spheroid section at equator following either 1, 3 or 5 hour incubation with 10 μg/ml Herceptin-FITC are shown for MDA and BT spheroids.

FIG. 4 depicts the surviving fraction of MCF7, MDA and BT cells in monolayer cultures following acute doses of external beam radiation.

FIG. 5 depicts the surviving fraction of MCF7, MDA and BT cells in monolayer cultures following 24 hr incubation with 3.7, 18.5 and 37 kBq/ml ²²⁵Ac labeled non-specific antibody.

FIG. 6 depicts spheroid response to external beam irradiation and increasing concentrations of ²²⁵Ac labeled non-specific antibody.

FIG. 7 depicts median growth curves for spheroids incubated 1 hr with 0.37, 1.85, 3.7, and 18.5 kBq/ml ²²⁵Ac on 10 μg/ml Herceptin, or 18.5 kBq/ml on non-specific antibody (hot control).

FIG. 8 depicts the growth of individual spheroids following 1 hr incubation with ²²⁵Ac-Herceptin.

FIG. 9 depicts microscope images of MDA spheroids 1, 21, 42 and 59 days post treatment.

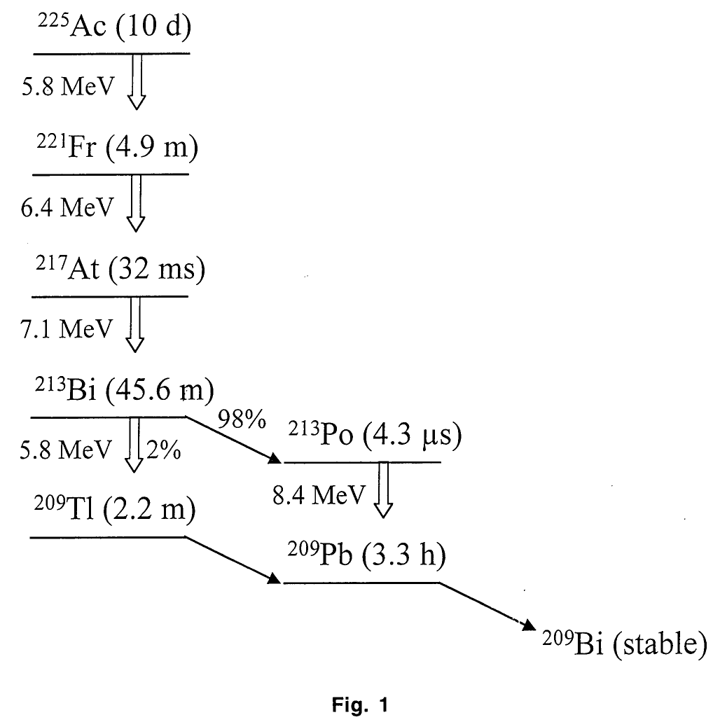

### DETAILED DESCRIPTION OF THE INVENTION

In one embodiment of the present invention, there is provided a method of preventing toxicity during radioimmunotherapy for intravascularly disseminated tumor cells in an individual comprising the steps of administering an antibody construct labeled with an alpha-particle emitting radionuclide where the antibody is specific for a protein expressed in the intravascular tumor cells; rapidly targeting the radiolabeled antibody construct to the intravascular tumor cells; internalizing the radiolabeled antibody conjugate into the intravascular tumor cells; and rapidly clearing the non-targeted intravascular radiolabeled antibody construct; whereby a combination of internalizing the radionuclide into the intravascular tumor cells and rapid clearance of the non-targeted intravascular radiolabeled antibody construct decreases alpha particle emission from the radionuclide and decay intermediates thereof to non-targeted cells thereby preventing systemic toxicity.

In an aspect of this embodiment, a representative disseminated cancer is breast cancer, a representative antibody is Herceptin and the expressed protein may be HER2/neu protein, and the radionuclide may be actinium-225 or radium-223. In all aspects of this embodiment, a representative range of time in which to target the radiolabeled antibody may be from about 2 hours to about 24 hours. The radionuclide may be administered in an amount of about 0.5 mCi to about 500 mCi. A representative range of time in which one would be able to rapidly clear the intravascular non-targeted radiolabeled antibody construct is about 2 hours to about 6 hours.

In another embodiment of this invention there is provided a method of targeting intravascularly disseminated breast cancer metastases for delivery of an alpha-particle emitting radionuclide thereto with minimal targeting of normal cells with said alpha-particle emitting radionuclide or alpha-particle emitting decay intermediates thereof in an individual, comprising the steps of conjugating the radionuclide with Herceptin antibody to form a radiolabeled Herceptin construct; administering the radiolabeled Herceptin construct intravenously to the individual; targeting the radiolabeled Herceptin construct to HER-2/neu protein expressed on the breast cancer metastases; internalizing the radiolabeled Herceptin construct into the breast cancer metastases; and rapidly clearing non-targeted intravascular radiolabeled Herceptin construct such that a combination of internalizing the radionuclide into the intravascular breast cancer metastases and rapid clearance of the non-targeted intravascular radiolabeled Herceptin construct minimizes targeting the alpha particle-emitting radionuclide and alpha-particle emitting decay intermediates thereof to normal cells. In all aspects of this embodiment the radionuclide used, the amount of radionuclide administered and the time to target the radiolabeled Herceptin construct or to rapidly clear intravascular non-target radiolabeled Herceptin construct is as disclosed supra.

The following definitions are given for the purpose of facilitating understanding of the inventions disclosed herein. Any terms not specifically defined should be interpreted according to the common meaning of the term in the art.

As used herein, the term “radiosensitivity parameter” as applied to the radiosensitivity of spheroids is the activity concentration required to reduce the treated to untreated spheroid volume ratio to 0.37. This is a measure of volume reduction and not a measure of cell sterilization.

As used herein, the term “volume reduction” refers to a number of biological variables including the rates of cellular sterilization, removal of sterilized cells, and cellular proliferation.

Provided herein is a treatment approach to minizmize systemic toxicity during radioimmunotherapy by using Herceptin labeled with the alpha particle emitting atomic generator, e.g., actinium-225, to rapidly target and to eradicate breast cancer metastases expressing variable levels of HER-2/neu and to rapidly clear intravascular nontargeted ²²⁵Ac-Herceptin antibodies. Rapid targeting of disseminated intravascular disease extends the scope of the antigen targets to include targets that are also expressed on the normal tissue from which the tumor is derived. Since the normal cells, such as breast cells, should not be found in the vasculature, any antibody that targets a protein on such normal cells can be useful because the antibody will be cleared before targeting of normal cells occurs. Thus, the need for an antibody to a specific tumor antigen is considerably lessened.

By rapidly removing the parent radionuclide, the continuous generation of such daughters is also removed which leads to a substantial reduction in the potential toxicity associated with free alpha-emitting daughters in the circulation. The ²²⁵Ac-Herceptin antibodies are administered intravenously to facilitate targeting. The radiolabeled Herceptin is allowed to target for about 2 hours to about 24 hours where it is subsequently internalized by the targeted cells.

Extracorporal immunoadsorption nor the use of clearing agents been considered for reduction of free alpha-emitting daughters from the vasculature. Furthermore, these approaches require a longer period of time or are dose limiting. However, implantation of an antigen-coated pellet would rapidly remove circulating antibody and the associated radionuclide and, by extension, decay intermediates, by concentrating the targeting antibody within the pellet. Non-targeted radiolabeled antibody clearance requires about 2 hours to about 6 hours. This provides both rapid clearance and eliminates potential toxicity to such organs as the kidneys.

In vitro a spheroid model represents rapidly accessible, intravascularly distributed tumor cell clusters. These results suggest, although not limited to said, that an ²²⁵Ac concentration in the range 9.25-18.5 kBq/ml (250-500 nCi/ml) should be sufficient to eradicate tumor cells with intermediate HER-2/nue expression. This translates to approximately 1 to 2 mCi for human administration. Based on animal studies, this activity concentration is clinically realistic when rapid removal of the circulating antibody is not performed. When the circulating antibody is rapidly removed, substantially greater amounts of radioactivity will be tolerated since, depending upon the elapsed time before removal, only a small fraction of the total amount of Ac-225 decays will have occurred in circulation. Thus a range of about 0.5 mCi to about 500 mCi is contemplated.

The response of spheroids to ²²⁵Ac-Herceptin is highly dependent on HER-2/neu expression. It is possible to sterilize spheroids with intermediate HER-2/neu expression and to induce a growth delay in low HER-2/neu expressing spheroids by increasing the specific activity of the radiolabeled antibody. A very high specificity relative to the hot controls was observed. Targeted spheroids are exposed to the atomic alpha-particle generator for a prolonged time period, corresponding to the average lifetime of ²²Ac, i.e., approximately 14 days, whereas exposure is limited to one hour in the hot control experiments. Longer hot control exposure durations such as the 24 hour period used in the radiosensitivity measurements showed volume reductions similar to those obtained with the 1 hour specific antibody incubation. The very high specificity seen with a short exposure time supports the rapid clearing strategy.

Methods of construction of radiolabeled constructs, e.g., antibodies and routes of administration of said are dependent on the disease targeted and are well-known to those or ordinary skill in the art. Additionally, such an artisan would be skilled in determining dosage and use of therapeutic radionuclides. As such, it is appreciated that the means and methods of use of radiolabeled antibodies for targeted radionuclide delivery with minimal concomitant systemic toxicity is not limited by the instant disclosure. It is contemplated that the rapid targeting/rapid clearing method disclosed herein would be applicable to, for example, targeting with the anti-prostate specific membrane (PSMA) antibody J591 for prostate cancer. Additionally, the invention is not limited to actinium-225 labeling. Other representative alpha-emitting radionuclides such as radium-223 can be used to label antibodies.

The following examples are given for the purpose of illustrating various embodiments of the invention and are not meant to limit the present invention in any fashion.

### EXAMPLE 1

### Cells

MCF7, MDA-MB-361 (MDA) and BT-474 (BT) were purchased from the American Type Culture Collection (ATCC, Manassas, Va.). MCF7 monolayer cultures were incubated in MEM with NEAA (MSKCC Media Lab, NY), MDA in L-15 (MSKCC Media Lab, NY), and BT in RPMI with 10 mM HEPES, 1 mM NA Pyruvate, 2mM L-Glutamine, 1.5 g/L Bicarbonate, and 4.5 g/L Glucose (MSKCC Media Lab, NY). The medium for all cell lines was supplemented with 10% FBS, 100 units/ml penicillin, and 100 μg/ml streptomycin. The cell cultures were kept at 37° C. in a humidified 5% CO₂ and 95% air incubator.

### EXAMPLE 2

### Flow Cytometry

The relative level of HER-2/neu expression for the three cell lines was determined using the Becton-Dickinson FACS Caliber Analyzer (Franklin Lakes, N.J.). Cells were first incubated with Herceptin for 0.5 hours, then washed and then incubated (0.5 h) with a fluorescently tagged antibody against the F_{c} portion of human IgG (Sigma, F-9512). A total of 10,000 events were collected. Relative expression levels, based upon the median values of the distributions were 1:4:15 for MCF7:MDA:BT.

### EXAMPLE 3

### Spheroids

Spheroids were initiated using the liquid overlay technique of Yuhas et al. Approximately 10⁶ cells, obtained by trypsinization from growing monolayer cultures, were seeded into 100 mm dishes coated with a thin layer of 1% agar (Bacto Agar, Difco, Detroit, Mich.) with 15 ml of medium. The medium used was the same as for monolayer cultures. After 5 to 7 days, spheroids of the MCF7 and MDA MB361 cell lines with approximate diameters of 200±20 μm were selected under an inverted phase-contrast microscope with an ocular scale using an Eppendorf pipette. The selected spheroids were transferred to 35 mm bacteriological petri dishes in 2 ml medium for treatment.

### EXAMPLE 4

### Antibodies

Herceptin (anti-HER-2/nue) (Genentech, Inc., South San Francisco, Calif.) was used as the specific antibody. HuM195 (anti-CD33) (Protein Design Laboratories, Inc. Sunnyview, Calif.) and J591 (anti-PSMA) (supplied by Dr. Neil Bander, Department of Urology, New York Presbyterian Hospital-Weill Medical College of Cornell University and Ludwig Institute for Cancer Research, New York, N.Y.) were used as non-specific controls.

### EXAMPLE 5

### Actinium-225 (²²⁵Ac)

²²⁵Ac was obtained from the Department of Energy (Oak Ridge National Laboratory, Oak Ridge, Tenn.) and was supplied as a dried nitrate residue. The ²²⁵Ac activity was measured with a Squibb CRC-17 Radioisotope Calibrator (E.R. Squibb and Sons, Inc., Princeton, N.J.) set at 775 and multiplying the displayed activity value by 5. The CRC-17 value was verified by counting an aliquot of the measured sample as a point source with pulse height multi-channel analysis (MCA) using an energy calibrated HPGe detector (Canberra Industries, Meriden, Conn.). The ²²⁵Ac nitrate residue was dissolved in 0.1 mL of 0.2 M Optima grade HCl (Fisher Scientific, Pittsburgh, Pa.). Metal-free water (MFW) used for this and all other solutions was obtained from a Purelab Plus system (U.S. Filter Corp., Lowell, Mass.) and was sterile filtered.

### EXAMPLE 6

### Reagents

Chemicals used in the radiolabeling and purification steps were ACS Reagent grade or better. Tetramethylammonium acetate (TMAA), ammonium acetate (NH₄Ac), sodium hydroxide (NaOH), 1-ascorbic acid (1-aa), sodium carbonate (Na₂CO₃), sodium hydrogen carbonate (NaHCO₃), Sephadex C-25, and diethylenetriaminepentaacetic acid (DTPA) were obtained from Aldrich Chemical Co (St Louis, Mo.). Human serum albumin (HSA) (Swiss Red Cross, Bern, Switzerland) and 0.9% NaCl (Abbott Laboratories, North Chicago, Ill.) were used as received. A 10 DG desalting column (Bio-Rad, Hercules, Calif.) was used as the stationary phase for the size exclusion chromatographic purification with a 1% HSA mobile phase.

### EXAMPLE 7

### Radiolabeling Methodology

The first step in construct preparation was the ²²⁵Ac-DOTA-NCS chelation reaction. The bifunctional isothiocyanoato-derived 2B-DOTA, 2-(p-isothiocyanatobenzyl)-1,4,7,10-tetraazacyclododecane-1,4,7,10-tetraacetic acid was obtained from Macrocyclics (Dallas, Tex.). Actinium-225 dissolved in 0.2 M HCl was mixed with 200 to 500 mg of 10 g/L DOTA-NCS in MFW, 0.015-0.020 mL of 150 g/L stock 1-ascorbic acid, and 0.025-0.150 mL of 2 M TMAA. The pH of the resulting reaction mixture was 5.5. The mixture was then heated to 60° C. for 30-45 min.

The second step in construct preparation was the ²²⁵Ac-DOTA-NCS reaction with the IgG. The ²²⁵Ac-DOTA-NCS chelation reaction was mixed with 0.5 to 1.0 mg of the IgG, 0.015-0.020 mL of 150 g/L stock 1-ascorbic acid, and 0.025-0.150 mL of a 1 M carbonate buffer. The pH of the resulting reaction mixture was 8.5 to 9.0. The reaction mixture was then heated to 36° C. for 30 to 60 min. At the end of the reaction period, the mixture was treated with a 0.020 mL addition of 10 mM DTPA to complex any free metals during the size exclusion chromatographic purification using a 10 DG size exclusion column with a 1% HSA as the mobile phase.

The radiochemical purity of ²²⁵Ac-DOTA-Herceptin was >90% as determined by instant thin layer chromatography methods and the immunoreactivity of the labeled product was between 70 and 80% as determined by cell based-assay methods (Nikula et al. J. Nucl. Med. Vol. 40, pp. 166 (1999)).

### EXAMPLE 8

### Antibody Penetration

Spheroids of diameter 200 μm were incubated with 10 μg/ml FITC (F7250, Sigma, St. Louis, Mo.) conjugated Herceptin for 1, 3 and 5 h and imaged by confocal microscope while still in the incubation medium. A 3 μm thick optical section was acquired at the center of each spheroid. Five spheroids were imaged for each time point. Antibody concentration as a function of radial distance was obtained using MIAU, a software package developed in-house. The method has been previously described.

Briefly, an erosion element is used to follow the exterior contour of each spheroid and the average pixel intensity in each ring is converted to antibody concentration by calibration with the known external concentration of antibody. The antibody concentration as a function of distance from the rim of the spheroid was corrected for light attenuation.

### EXAMPLE 9

### Radiosensitivity of Cell Lines Determined in Monolayer Culture and in Spheroids

The radiosensitivity of the different cell lines was determined in monolayer cultures using the colony forming assay. Depending on the radiation dose, between 10³ and 10⁷ cells were plated in monolayer cultures. External beam radiosensitivity was determined following exposure to acute doses of 3, 6, 9, or 12 Gy photon irradiation using a cesium irradiator at a dose rate of 0.8 Gy/min (Cs-137 Model 68, JL Shepherd and Associates, Glendale, Calif.). The absorbed dose required to yield a 37% survival, i.e., the D₀ value) was obtained by fitting a monoexponential function to the log-linear portion of the surviving fraction curve. Monolayer cultures incubated with 3.7, 18.5, and 37 kBq/ml ²²⁵Ac-labeled non-specific antibody for 24 hr were used to determine alpha-particle radiosensitivity. Over a 24-hr period, 6.7% of the total number of ²²⁵Ac atoms will have decayed. Since the longest lived daughter, Bi-213, has a half-life of 45.6 min, all daughters generated during this period will also decay. Assuming, therefore, that each decay of ²²⁵Ac deposits the sum of all 4 alpha-particle energies, the absorbed dose is estimated to be 1.4, 6.8, and 13.6 Gy for each of the three concentrations, respectively.

The radiosensitivity of spheroids was evaluated as the activity concentration required to reduce the treated to untreated spheroid volume ratio to 0.37. Since this parameter depends upon the day post-therapy, volume ratios from day 20 to day 45 post-therapy were examined and the median across this range was used. By plotting this volume ratio versus activity concentration and fitting the log-linear portion of the curve to a monoexponential function, a radiosensitivity parameter may be derived from the slope. The inverse of the slope gives the dose that yields a volume ratio of 0.37. This value is denoted DVR₃₇, and it is loosely analogous to the D₀ in colony formation assays.

### EXAMPLE 10

### Uptake of Herceptin by MDA and BT Spheroids

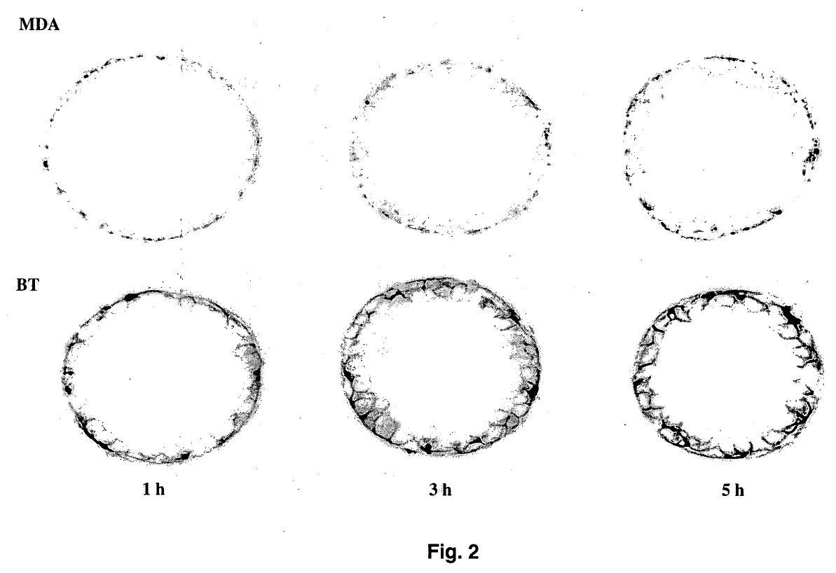

Penetration of Herceptin into spheroids of cells with different HER-2/neu expression levels was evaluated by measuring FITC labeled Herceptin by confocal microscopy. Images acquired through the equator of 200 μm diameter spheroids incubated for 1, 3 and 5 hours with 10 μg/ml Herceptin-FITC are shown for MDA and BT spheroids in FIG. 2. The cells on the spheroid rim are clearly outlined consistent with cell-surface localization of HER-2/neu. Herceptin has penetrated approximately 1, 2 and 3 cell layers after 1, 3 and 5 hours incubation, respectively. FITC intensity was converted to antibody concentration as described in the methods.

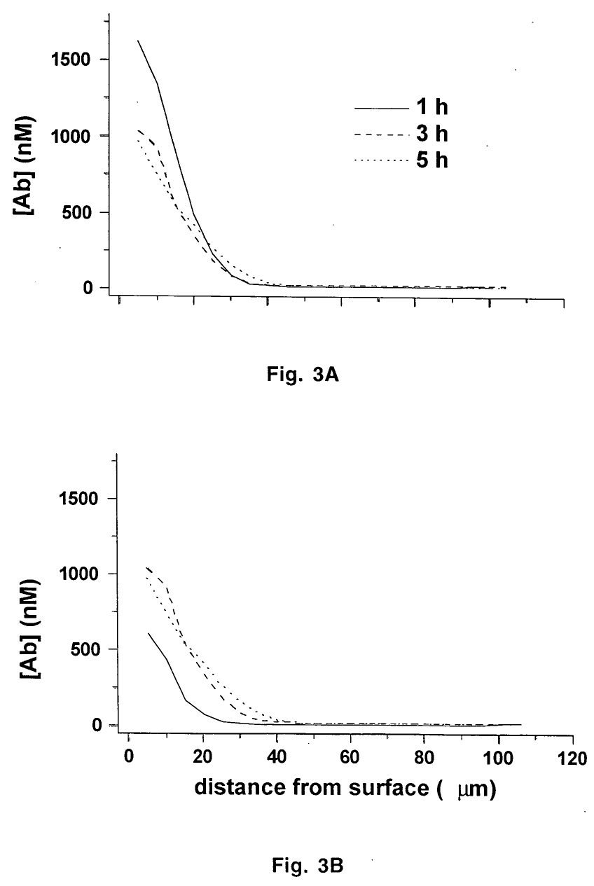

The results are depicted in FIG. 3. Each curve corresponds to the median of 5 individual spheroid measurements. The dose-response for unlabeled Herceptin was evaluated by incubating spheroids of the 3 cell lines for 1 hour in 10, 50, 100 and 500 μg/ml Herceptin. No impact upon spheroid growth was observed (data not shown).

The confocal microscopy images of Herceptin concentration profiles show a slightly deeper penetration in the BT spheroids than in the MDA spheroids. The Herceptin concentration at the surface of the BT spheroids after 1 hr incubation is found to be a factor 2-3 higher than for the MDA spheroids while the penetration depth into the spheroids were similar. This is in good agreement with the relative HER-2/neu expression of 15:4 for BT:MDA. The range of alpha particles is typically 80-100 μm and a complete penetration of the antibody is not required to deliver radiation to the spheroid core. Thus, a 10 μg/ml Herceptin concentration is adequate for spheroid kill.

Herceptin incubation in monolayer cultures is reported to result in increased cell doubling time leading to an increase in cell dormancy. This was not observed in spheroids where a 1-hour incubation with concentrations up to 500 μg/ml Herceptin had no effect on spheroid growth kinetics for the three cell lines tested. The absence of an effect on spheroids as opposed to monolayer cultures is probably the result of incomplete penetration, increased resistance to cytotoxic and growth inhibitory agents of spheroids relative to monolayer cultures.

### EXAMPLE 12

### Radiosensitivity of MCF7, MDA and BT Cells

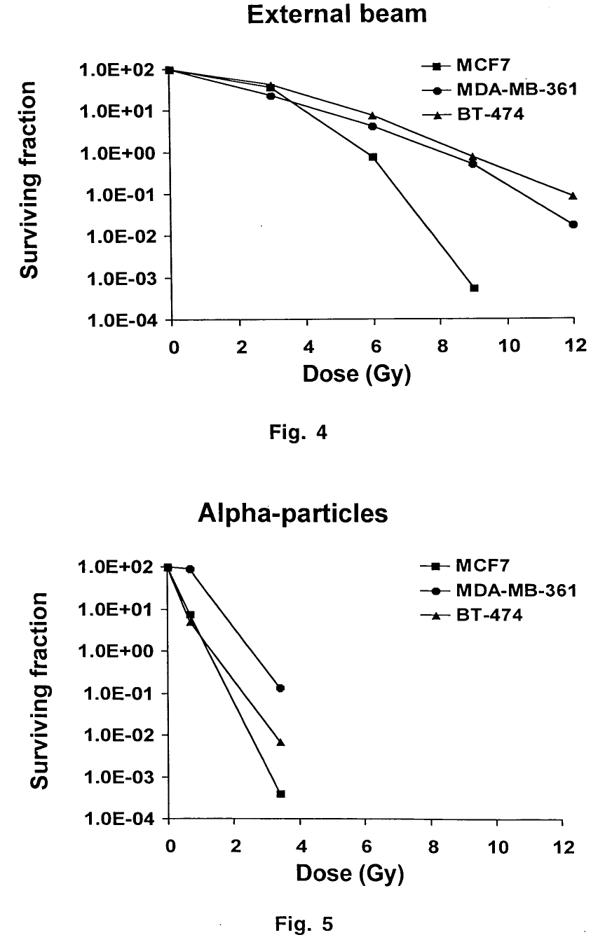

The radiosensitivity of the of MCF7, MDA and BT cell lines was determined using the colony forming assay described above. To discriminate between inherent radiosensitivity of the different cell lines and increased targeting due to the differential expression of HER-2/neu, the radiosensitivity of each cell line was determined in monolayer cultures after external beam irradiation and after incubation with ²²⁵Ac labeled non-specific antibody. The surviving fraction of cells in monolayer culture is plotted versus absorbed photon dose in FIG. 4 and absorbed alpha particle dose in FIG. 5. The dose required to reduce the surviving fraction to 37%, D₀, is listed in Table 1.

TABLE 1
Dose, D₀, required to reduce surviving fraction of cells in
monolayer cultures following external beam and alpha-particle
irradiation to 37%.

| | External beam | Alpha-particle |
|---|---|---|
| Cell line | D₀ (Gy) | D₀ (Gy) |
| MCF7 | 0.7 | 0.3 |
| MDA | 1.4 | 0.5 |
| BT | 1.7 | 0.4 |

In monolayer cultures, the two HER-2/nue positive cell lines MDA and BT are approximately equivalent in photon radiosensitivity to low-LET, high-dose rate external beam radiation, whereas MCF7 is approximately 2-fold more radiosensitive with D₀ values of 0.7, 1.4 and 1.7 Gy, respectively. MCF7 is also the most sensitive line to alpha-particle irradiation, with MDA having the lowest sensitivity to alphas of the three and BT alpha sensitivity falling between the other two cell lines. The corresponding alpha-D₀ values are 0.3, 0.5, and 0.4 Gy.

### EXAMPLE 13

### Effect of External Beam Radiation and ²²⁵Ac Labeled Non-specific Antibody on Spheroids

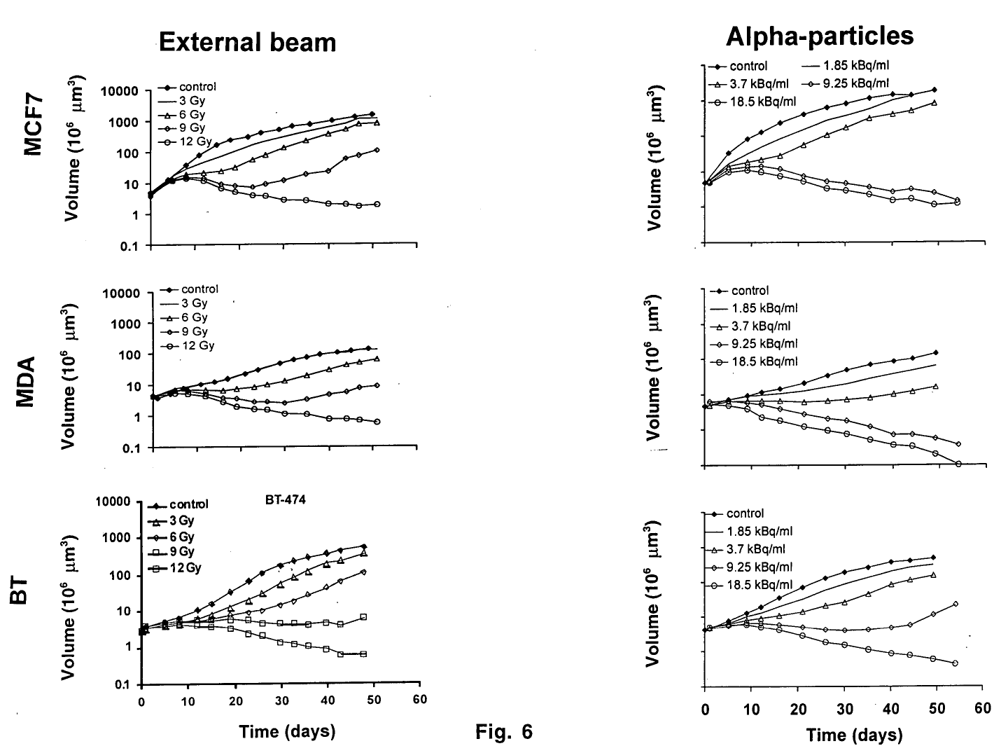

As with the monolayer cultures the radiosensitivity of spheroids was determined. Spheroid response to 3, 6, 9, and 12 Gy external beam irradiation and increasing concentrations of ²²Ac labeled non-specific antibody (24 h incubation) is depicted in FIG. 6. At 12 Gy, outgrowth assays for MCF7 and BT spheroids showed viable cells, whereas no colonies were formed for MDA spheroids. At the two highest concentrations of non-specific radiolabeled antibody, outgrowth assays for MCF7 and MDA spheroids yielded no colonies; for BT the same result was obtained only at the highest radioactivity concentration used.

The dose required to reduce the volume ratio of treated to untreated spheroids to 0.37, DVR₃₇, was used as a measure of spheroid radiosensitivity and is listed in Table 2. Spheroids were found to have a greater differential sensitivity to alpha-particles than to external beam irradiation even though the opposite is true in monolayer cultures. MCF7, MDA and BT spheroids were found to have similar external beam radiosensitivity. An external beam radiosensitivity of 2 Gy was found for spheroids of all three cell lines. Following alpha-particle irradiation a DVR₃₇ of 1.5, 3.0, and 2.2 kBq/ml was determined for MCF7, MDA, and BT, respectively.

TABLE 2
Dose required to reduce the treated to untreated spheroid volume
ratio to 0.37

| | External beam | Alpha-particle |
|---|---|---|
| Cell line | DVR₃₇ (Gy) | DVR₃₇ (kBq/ml) |
| MCF7 | 2 | 1.5 |
| MDA | 2 | 3.0 |
| BT | 2 | 2.2 |

### EXAMPLE 14

### Determination of Spheroid Growth in the Presence of ²²⁵Ac-Herceptin

The response to ²²⁵Ac labeled Herceptin was evaluated by incubating spheroids with 0.37, 1.85, 3.70, or 18.50 kBq/ml ²²⁵Ac on 10 μg/ml Herceptin (specific antibody) for 1 hour. Spheroids exposed to 18.50 kBq/ml ²²⁵Ac on 10 μg/ml irrelevant antibody (hot control), 10 μg/ml unlabeled Herceptin (cold control) and untreated spheroids (control) were followed in the same manner. Twenty-four or twelve spheroids were used in each experiment. After incubation, the spheroids were washed three times by suspension in fresh medium and placed in separate wells of a 24-well plate. The media in each well was replaced and individual spheroid volume measurements were performed twice per week. The concentrations used in these studies translate to human administered activities in the mCi to sub-mCi range.

An inverted phase microscope fitted with an ocular micrometer was used to determine the major and minor diameter dₘₐₓ and dₘᵢₙ, respectively, of each spheroid. Spheroid volume was calculated as V=π·dₘₐₓ·dₘᵢₙ²/6. Volume monitoring was stopped once a spheroid diameter exceeded 1 mm or when the spheroid fragmented to individual cells or smaller (2- to 3-cell) clusters. The viability of such fragments was assessed in an outgrowth assay by plating the cell clusters on to adherent dishes, incubating for 2 weeks, and then evaluating for colony formation or outgrowth.

### EXAMPLE 15

### Effect of ²²⁵Ac-Herceptin on Spheroid Growth

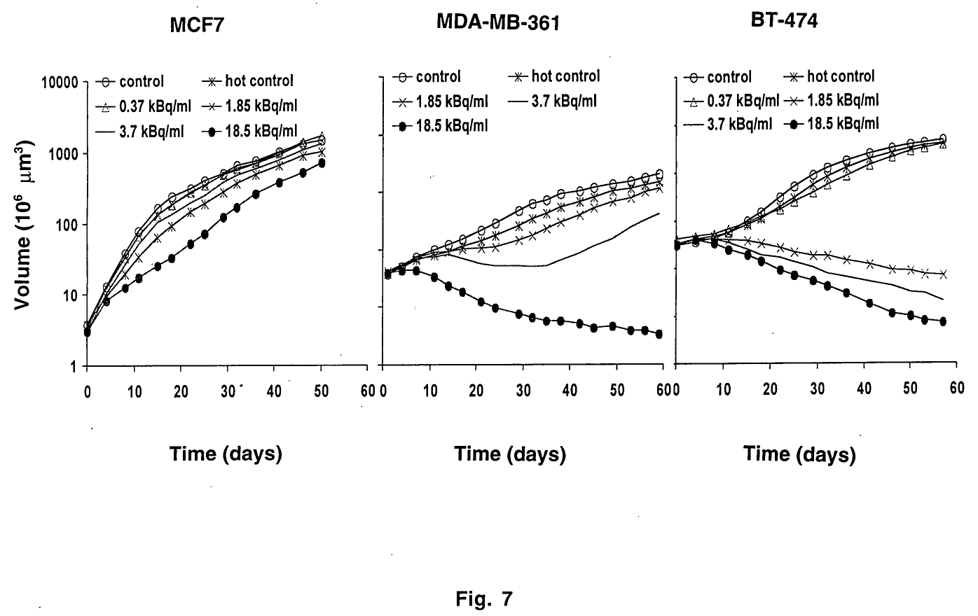

Treatment efficacy was assessed from spheroid growth curves as described above. Median growth curves for MCF7, MDA and BT spheroids are depicted in FIG. 7. At day 35, the median volume of spheroids treated with 18.5 kBq/ml ²²⁵Ac-Herceptin relative to untreated control spheroids was 34%, 0.8% and 0.2% for MCF7, MDA and BT, respectively. The corresponding values for spheroids treated with ²²⁵Ac labeled non-specific antibody (hot control) were 65%, 58% and 71%, respectively. The ²²⁵Ac activity concentration required to yield a 50% reduction in spheroid volume relative to untreated spheroids at day 35 was 11.8, 1.1 and 0.4 kBq/ml (320, 30, 10 nCi/ml) for MCF7, MDA and BT spheroids, respectively.

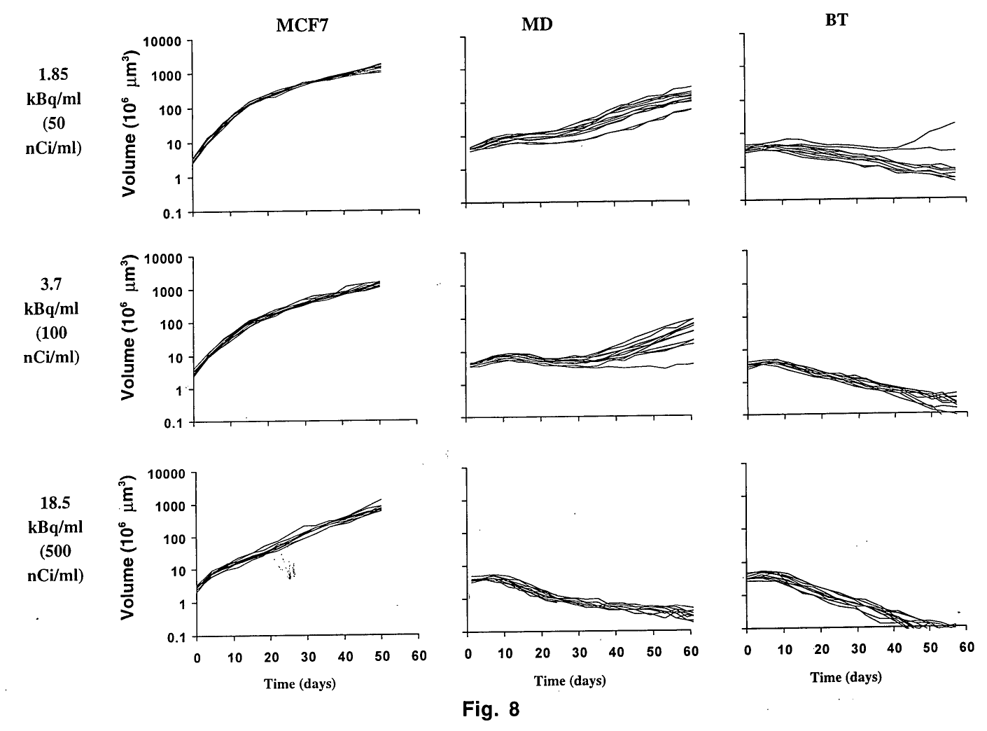

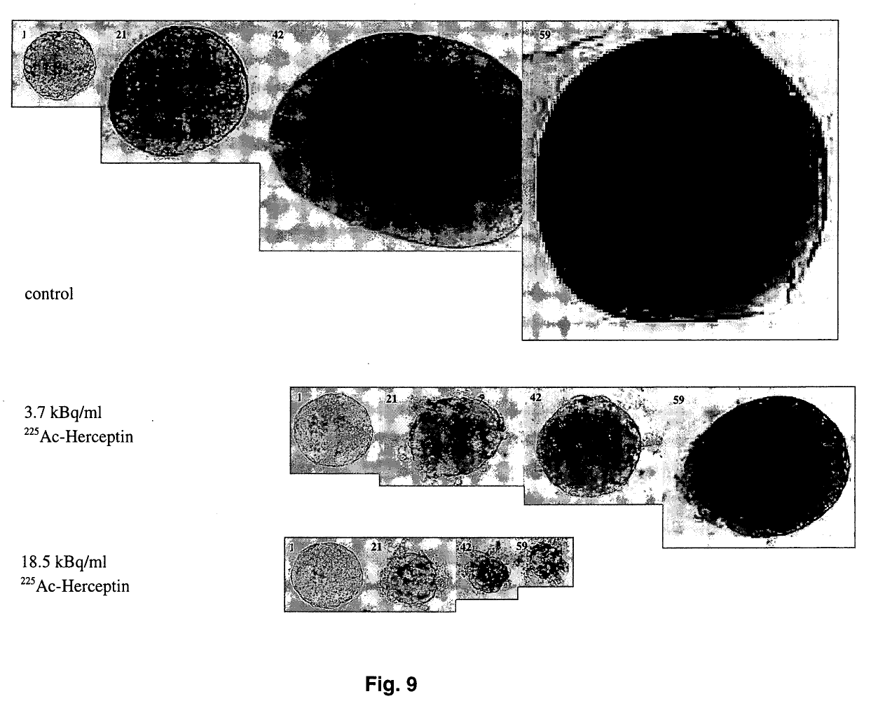

Growth of individual spheroids following 1 hr incubation with increasing concentrations of ²²⁵Ac on 10 μg/ml Herceptin are shown in FIG. 8. MCF7 spheroids continued growing but with a 20 to 30 day growth delay at 18.5 kBq/ml. MDA spheroid growth was delayed by 30 to 40 days at 3.7 kBq/ml. At 1.85 kBq/ml activity concentration 2 of 12 BT spheroids were viable; no colonies were observed at 3.7 and 18.5 kBq/ml for this cell line. At 18.5 kBq/ml, 12/12 spheroids disaggregated after 70 days and cells remaining from each spheroid failed to form colonies within 2 weeks of being transferred to adherent dishes. All BT spheroids at activity concentrations >3.7 kBq/ml failed to regrow and to form colonies. Likewise, no colonies were observed for MDA spheroids treated at 18.5 kBq/ml. FIG. 9 depicts optical microscope images of MDA spheroids following ²²⁵Ac-Herceptin treatment.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of preventing toxicity during radioimmunotherapy for intravascularly disseminated tumor cells in an individual comprising the steps of:
administering an antibody construct labeled with an alpha-particle emitting radionuclide, said radiolabeled antibody specific for a protein expressed in said intravascular tumor cells;
rapidly targeting said radiolabeled antibody construct to said intravascular tumor cells;
internalizing said radiolabeled antibody conjugate into said intravascular tumor cells; and
rapidly clearing non-targeted intravascular radiolabeled antibody construct, wherein a combination of internalizing the radionuclide into said intravascular tumor cells and rapid clearance of said non-targeted intravascular radiolabeled antibody construct decreases alpha particle emission from the radionuclide and decay intermediates thereof to non-targeted cells thereby preventing systemic toxicity.

2. The method of claim 1, wherein said disseminated tumor cells are breast cancer metastases.

3. The method of claim 1, wherein said antibody is Herceptin.

4. The method of claim 1, wherein said alpha-emitter is Ac-225 or Ra-223.

5. The method of claim 1, wherein said expressed protein is HER-2/neu.

6. The method of claim 1, wherein targeting said radiolabeled antibody construct takes from about 2 hours to about 24 hours.

7. The method of claim 1, wherein said radionuclide is administered in an amount of about 0.5 mCi to about 500 mCi.

8. The method of claim 1, wherein rapid clearance of said intravascular non-targeted radiolabeled antibody construct requires about 2 hours to about 6 hours

9. A method of targeting intravascularly disseminated breast cancer metastases for delivery of an alpha particle-emitting radionuclide thereto with minimal targeting of normal cells with said alpha particle-emitting radionuclide or alpha-particle emitting decay intermediates thereof in an individual, comprising the steps of:
conjugating the radionuclide with Herceptin to form an antibody construct;
administering said radiolabeled Herceptin construct intravenously to the individual;
targeting said radiolabeled Herceptin construct to HER-2/neu protein expressed on said breast cancer metastases;
internalizing said radiolabeled Herceptin construct into said breast cancer metastases; and
rapidly clearing non-targeted intravascular radiolabeled Herceptin construct;
wherein a combination of internalizing the radionuclide into said intravascular breast cancer metastases and rapid clearance of said non-targeted intravascular radiolabeled Herceptin construct minimizes targeting alpha particle-emitting radionucled and alpha-particle emitting decay intermediates thereof to normal cells.

10. The method of claim 9, wherein said alpha-emitter is Ac-225 or Ra-223.

11. The method of claim 9, wherein targeting said 225Ac-Herceptin construct takes from about 2 hours to about 24 hours.

12. The method of claim 9, wherein said radionuclide is administered in an amount of about 0.5 mCi to about 500 mCi.

13. The method of claim 9, wherein rapid clearance of said intravascular non-targeted radiolabeled Herceptin construct requires about 2 hours to about 6 hours.
